(19) United States
(12) Patent Application Publication
(10) Pub. No.: US 2022/0046914 A1
(43) Pub. Date: Feb. 17, 2022

(54) DIFFUSER OF VOLATILE SUBSTANCES BY COMBUSTION

(71) Applicant: ZOBELE HOLDING SPA, Trento (IT)

(72) Inventors: Cedric MORHAIN, Barcelona (ES); Montserrat RIERA GINER, Barcelona (ES)

(21) Appl. No.: 17/417,179

(22) PCT Filed: Dec. 23, 2019
(86) PCT No.: PCT/EP2019/086934

§ 371 Date: Jun. 22, 2021

(30) Foreign Application Priority Data
Dec. 26, 2018 (ES) P201831279

(51) Int. Cl. A01N 25/20; A61L 9/02; A01N 65/44; A01N 31/02; A01N 53/00; A01N 25/08; A01P 17/00

(52) U.S. Cl. CPC A01N 25/20; A61L 9/02; A01N 65/44; A01P 17/00; A01N 53/00; A01N 25/08; A01N 31/02

(57) ABSTRACT

The diffuser of volatile substances by combustion comprises a body (1) that includes said volatile substances, wherein said body is formed from microcapsules (2) that house said volatile substances. Preferably, each microcapsule (2) comprises an outer shell that houses the volatile substances therein. It makes it possible to provide a diffuser of volatile substances by combustion wherein the evaporation of the volatile substances is reduced or fully prevented during the drying in the manufacturing process thereof.

The present invention relates to a diffuser of volatile substances by combustion, in particular, to a coil-shaped diffuser for repelling insects, for example, mosquitoes, or to a diffuser used as an air freshener.

### BACKGROUND OF THE INVENTION

Mosquito coils used as insecticides have been known for many years. Despite this, they are still a widely used product, particularly for external applications.

In general, they consist of a mixture of wet-extruded wood particles with the volatile active ingredient and also generally with an additive to control flameless combustion. After the extrusion process, the extruded profile is exposed to a forming finishing process and is then dried in order to obtain a solid product.

The drying process is a highly sensitive and critical process. An excessively high temperature during drying may cause a portion of the active ingredient to evaporate, reducing the future efficiency of the coil, in addition to economic loss, since the active ingredient is normally considerably more expensive than the other component of the mixture.

Additionally, the final content of the active ingredient is of great importance, since this value is usually indicated in the health registry of biocidal products with a certain permitted tolerance, and falling outside this tolerance may entail the withdrawal of the product from the market.

Therefore, drying generally takes place over a considerably long period of time, closer to days than hours, at a very low temperature of 40-80° C. to prevent the evaporation of the active ingredients of standard insecticides, which usually have low volatility.

Over the past decades, the insecticide active ingredients industry has striven to obtain new molecules of active ingredients with lower toxicity, greater efficiency and also higher volatility.

The current mosquito coil manufacturing process that entails wet extrusion and subsequent drying is completely inadequate for this new generation of insecticides, since they evaporate significantly at the temperature applied during drying.

Moreover, microencapsulation is a well-known process and the use thereof for reducing the evaporation of highly volatile substances is quite common. A typical example is microencapsulated fragrance that is mechanically ruptured, which is a capsule wherein the membrane surrounds a drop of fragrance and prevents the release thereof provided that the user does not break it applying pressure thereto.

It is typically applied in scratch and sniff labels, to allow the user to smell the product before buying it, detergents and fabric softeners, for a lasting effect of the fragrance in the fabric, etc. For these applications, the most common type of microcapsules used is based on a melamine membrane.

This type of capsules could meet the need to prevent the loss of active ingredients during drying, but would barely survive the mechanical stress generated during an extrusion process. Therefore, an objective of the present invention is to provide a diffuser of volatile substances by combustion wherein the evaporation of the volatile substances is reduced or completely avoided during the drying in the manufacturing process thereof.

### DESCRIPTION OF THE INVENTION

The cited drawbacks are addressed by the diffuser of the invention, which has other advantages that will be described below.

The diffuser of volatile substances by combustion in accordance with the present invention comprises a body that includes said volatile substances, wherein said body is formed from microcapsules that house said volatile substances.

Advantageously, each microcapsule comprises an outer shell that houses the volatile substances therein, and wherein the outer shell can be made of a polymer material, and/or is chosen from the group of gum arabic, xanthan gum, fatty acids, fatty alcohols, natural waxes and polyethylene.

Additionally, said outer shell is made of a material that is thermally activated, such that it melts, expands or collapses above a preset temperature, diffusing the volatile substances in the ambient air. For example, said outer shell is thermally activated at a temperature comprised between 80° C. and 150° C.

Preferably, said outer shell houses, together with the active substances, sawdust, combustion-assisting products, antioxidants and/or antifungal agents, although it may also house conventional components of this type of diffusers of volatile substances by combustion therein.

According to a preferred embodiment, said sawdust has a particle size comprised between 0.1 and 1 mm, the combustion-assisting products are chosen from the group comprising sodium nitrate, potassium nitrate and sodium benzoate, and said volatile substances are chosen from the group comprising pyrethroids, geraniol and/or citronella.

According to a preferred embodiment, said body has a coil shape.

Therefore, in the diffuser of the present invention, such as, for example, a mosquito coil, the volatile substances are not added like a liquid, but rather are housed in microcapsules, and said microcapsules reduce or completely avoid the evaporation of the active ingredient during drying.

### BRIEF DESCRIPTION OF THE FIGURES

In order to better understand the foregoing, drawings are provided wherein, schematically and solely by way of non-limiting example, a practical embodiment is represented.

FIG. 1 shows a perspective view of the diffuser of volatile substances by combustion according to an embodiment of the present invention, wherein the microcapsules are represented in a portion thereof.

### DESCRIPTION OF A PREFERRED EMBODIMENT

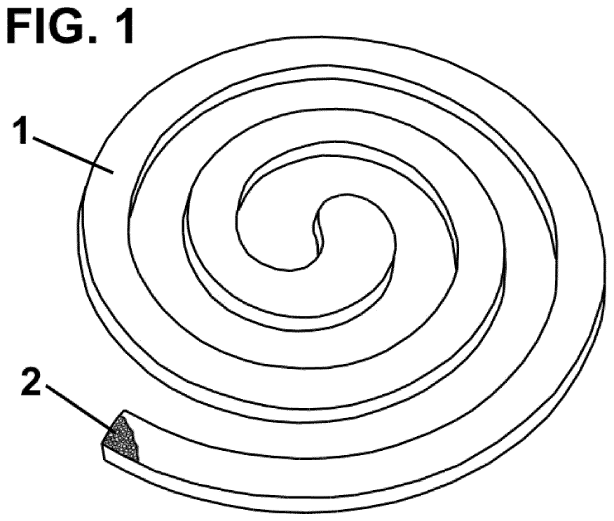

As shown in FIG. 1, the diffuser of volatile substances by combustion according to the present invention comprises a body 1, for example, coil-shaped according to the represented embodiment that includes the volatile substances to be diffused in the ambient air, wherein said volatile substances may be insecticidal in order to repel insects or aromatic agents in order to perfume the ambient air.

According to the present invention, the body 1 is formed from microcapsules 2 that house the volatile substances therein, in particular, by means of a polymer outer shell.

For use, said body 1 is burned, causing the outer shell of the microcapsules 1 to rupture due to temperature and diffuse the volatile substances in the ambient air.

Therefore, the type of microcapsules of interest to the present invention are temperature-activated microcapsules, i.e., they rupture when a preset temperature is exceeded, not those which are mechanically activated or ruptured by friction, as in conventional microcapsules.

Activation can be achieved through two different mechanisms: temperature-sensitive release, wherein the materials of the shell expand or collapse at a specific critical temperature, and fusion-activated release, wherein the material of the shell melts due to the increase in temperature.

The materials of the shell with such properties may be (but is not limited to), arabic gum, xanthan gum, fatty acids, fatty alcohols, natural waxes, polyethylene, etc.

Therefore, the polymer shell will protect the volatile substances and prevent the evaporation thereof during drying, and will rupture or melt when exposed to combustion heat and release the volatile substances in the ambient air.

Although microcapsule activation mechanisms are very fast, the increase in temperature when the combustion front comes closer to the microcapsules may be equally as good.

Consequently, it is critical that the volatile substance be released sufficiently fast to prevent the chemical integrity thereof from being affected by the temperature.

Therefore, it is preferable for the thermal activation of the microcapsules to fall within the temperature range of 80° C. to 150° C.

Regarding the composition of the contents of the microcapsules, they comprise:
sawdust with a particle size of 0.1 to 1 mm,
volatile substances in the range of 0.05 to 5% by weight (0.05%, considering next-generation highly efficient pyrethroids), 5% considering low-performance insecticides such as geraniol or citronella,
combustion-assisting products between 0.2 and 1% by weight (the combustion-assisting products may be sodium nitrate, potassium nitrate, sodium benzoate or any other material commonly used as to assist combustion),
antioxidants, and
antifungal agents.

Advantageously, the microcapsules will be added prior to extrusion as a suspension. The suspension is an aqueous suspension of microcapsules, generally having a maximum concentration of microcapsules of approximately 50%.

As explained earlier, for the extrusion of the body of the diffuser, water is added to the mixture of water with additives. Therefore, if a suspension is used, the percentage of microcapsules will be approximately equal to the equivalent percentage without the microcapsules, but we will eliminate the equivalent of the water in the suspension from the amount of added value.

Despite the fact that reference has been made to a specific embodiment of the invention, it is evident for a person skilled in the art that the described diffuser is susceptible to numerous variations and modifications, and that all the aforementioned details may be replaced by other, technically equivalent ones, without falling outside the scope of protection defined by the attached claims.

1. A diffuser of volatile substances by combustion, comprising a body (1) that includes said volatile substances, characterised in that said body is formed from microcapsules (2) that house said volatile substances.

2. The diffuser of volatile substances by combustion, according to claim 1, wherein each microcapsule (2) comprises an outer shell that houses the volatile substances therein.

3. The diffuser of volatile substances by combustion, according to claim 2, wherein the outer shell is made of a polymer material.

4. The diffuser of volatile substances by combustion, according to claim 2, wherein the material of said outer shell is chosen from the group of arabic gum, xanthan gum, fatty acids, fatty alcohols, natural waxes and polyethylene.

5. The diffuser of volatile substances by combustion, according to claim 2, wherein said outer shell is made of a material that melts, expands or collapses above a preset temperature.

6. The diffuser of volatile substances by combustion, according to claim 5, wherein said preset temperature is comprised between 80° C. and 150° C.

7. The diffuser of volatile substances by combustion, according to claim 2, wherein said outer shell houses, together with the active substances, sawdust, combustion-assisting products, antioxidants and/or antifungal agents therein.

8. The diffuser of volatile substances by combustion, according to claim 7, wherein said sawdust has a particle size comprised between 0.1 and 1 mm.

9. The diffuser of volatile substances by combustion, according to claim 7, wherein the combustion-assisting products are chosen from the group comprising sodium nitrate, potassium nitrate and sodium benzoate.

10. The diffuser of volatile substances by combustion, according to claim 1, wherein said volatile substances are chosen from the group comprising pyrethroids, geraniol and/or citronella.

11. The diffuser of volatile substances by combustion, according to claim 1, wherein said body (1) has a coil shape.
